(19) United States
(12) Patent Application Publication
(10) Pub. No.: US 2005/0031572 A1
(43) Pub. Date: Feb. 10, 2005

(54) Cosmetic or dermatological preparations including hops or hop-malt extracts and methods of using same for the prophylaxis and treatment of skin symptoms

(75) Inventors: Stefan Gallinat, Wedel (DE); Kirsten Venzke, Hamburg (DE); Thomas Blatt, Wedel (DE); Helga Biergiesser, Reinbek (DE); Thomas Doring, Hamburg (DE); Franz Stab, Echem (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/749,626

(22) Filed: Dec. 31, 2003

Related U.S. Application Data
(63) Continuation of application No. PCT/EP02/07388, filed on Jul. 3, 2002.

(30) Foreign Application Priority Data
Jul. 6, 2001 (DE) 101 32 953.9

(51) Int. Cl. A61K 7/06; A61K 35/78

(52) U.S. Cl. 424/74; 424/778

(57) ABSTRACT

The invention is a cosmetic or dermatological preparation that includes hops or hop-malt extracts and to methods of using this preparation for the prophylaxis and treatment of the symptoms that result from certain skin conditions such as skin disease, inflammatory skin conditions and degenerative skin conditions.

### CROSS-REFERENCE TO RELATED APPLICATIONS

This is a continuation application of PCTEP02/07388, filed Jul. 3, 2002, which is incorporated herein by reference in its entirety, and also claims the benefit of German Priority Application No. 101 32 953.9, filed Jul. 6, 2001.

### FIELD OF THE INVENTION

The present invention relates to the use of hop extracts or hop-malt extracts in cosmetic or dermatological preparations for the treatment and prophylaxis of the symptoms of negative changes in the skin caused by the environment, e.g. by skin damage induced by UV and/or ozone and/or smog and/or smoking, and of inflammatory and degenerative skin conditions.

### BACKGROUND OF THE INVENTION

Cosmetic skin care is primarily understood as meaning that the natural function of the skin as a barrier against environmental influences (e.g. dirt, chemicals, microorganisms) and against the loss of substances intrinsic to the body (e.g. water, natural fats, electrolytes) is strengthened or restored.

Impairment of this function may lead to increased resorption of toxic or allergenic substances or to attack by microorganisms, resulting in toxic or allergic skin reactions.

Another aim of skin care is to compensate for the loss by the skin of sebum and water caused by daily washing. This is particularly important if the natural regeneration ability is inadequate. Furthermore, skincare products should protect against environmental influences, in particular against sun and wind, and delay skin aging.

Chronological skin aging is caused, for example, by endogenous, genetically determined factors. The following structural damage and functional disorders, which can also come under the term “senile xerosis”, arise, for example, in the epidermis and dermis as the result of aging:
a) Dryness, roughness and formation of dryness wrinkles,
b) Itching and
c) Reduced refatting by sebaceous glands (e.g. after washing).

Exogenous factors, such as UV light and chemical noxae, can have a cumulative effect and, for example, accelerate or supplement the endogenous aging processes. In the epidermis and dermis, for example, the following structural damage and functional disorders may arise in the skin in particular as a result of exogenous factors; these are more far-reaching than the degree and quality of the damage in the case of chronological aging:
d) Visible vascular dilation (teleangiectases, cuperosis);
e) Flaccidity and formation of wrinkles;
f) Local hyperpigmentation, hypopigmentation and abnormal pigmentation (e.g. age spots) and
g) Increased susceptibility to mechanical stress (e.g. cracking).

The present invention relates in particular to products for the care of skin stressed by the environmental noxae, such as, for example, UV light, ozone, cigarette smoke, and also for the treatment of the damage caused by photoaging, in particular of the phenomena listed under a) to g).

Products for the care of aged skin are known per se. They comprise, for example, retinoids (vitamin A acid and/or derivatives thereof) or vitamin A and/or derivatives thereof. Their effect on structural damage is, however, limited. Furthermore, in product development, there are considerable difficulties in stabilizing the active ingredients to an adequate extent against oxidative decay. The use of products containing vitamin A acid, moreover, often causes severe erythematous skin irritations. Retinoids can therefore only be used in low concentrations.

In particular, the present invention relates to cosmetic preparations having effective protection against harmful oxidation processes in the skin, but also for the protection of cosmetic preparations themselves or for the protection of the constituents of cosmetic preparations against harmful oxidation processes.

The harmful effect of the ultraviolet part of solar radiation on the skin is generally known. The rays have various effects on the skin as an organ depending on their particular wavelength; UV-C radiation with a wavelength below 290 nm is absorbed by the ozone layer in the earth's atmosphere and therefore has no physiological significance. By contrast, rays in the range between 290 nm and 320 nm, the UV-B range, cause erythema, simple sunburn or even more or less severe burns. A maximum for the erythema activity of sunlight is stated to be the narrower range around 308 nm.

Numerous compounds are known for protecting against UV-B radiation, examples thereof being derivatives of 3-benzylidenecamphor, of 4-aminobenzoic acid, of cinnamic acid, of salicylic acid, of benzophenone, and of s-triazine.

It has long been incorrectly assumed that the long-wavelength UV-A radiation with a wavelength between 320 nm and 400 nm has only a negligible biological effect. However, it has now been proved by numerous studies that UV-A radiation is far more hazardous than UV-B radiation with regard to the triggering of photodynamic, specifically phototoxic, reactions and chronic changes in the skin. The harmful effect of UV-B radiation can also be further intensified by UV-A radiation.

Thus, it has been proved, inter alia, that even UV-A radiation under entirely normal everyday conditions is sufficient to damage within a short time the collagen and elastin fibers which are of essential importance for the structure and firmness of the skin. This results in chronic light-induced skin changes—the skin “ages” prematurely. The clinical appearance of skin aged by light includes, for example, wrinkles and lines and an irregular, furrowed relief. In addition, the areas affected by light-induced skin aging may have irregular pigmentation. The formation of brown spots, keratoses and even carcinomas or malignant melanomas is also possible. Skin aged prematurely by everyday exposure to UV is additionally characterized by a lower activity of the Langerhans cells and a slight chronic inflammation.

About 90% of the ultraviolet radiation that reaches the earth consists of UV-A rays. Whereas UV-B radiation varies greatly depending on a large number of factors (for example season and time of day or latitude), UV-A radiation remains relatively constant from day to day irrespective of seasonal and diurnal or geographic factors. At the same time, most of the UV-A radiation penetrates into the living epidermis, while about 70% of UV-B rays are retained by the horny layer.

It is therefore of fundamental importance that cosmetic and dermatological light protection preparations provide adequate protection both against UV-B and against UV-A radiation.

UV radiation can, however, also lead to photochemical reactions, in which case the photochemical reaction products then intervene in the skin's metabolism. In addition, UV radiation is a type of ionizing radiation. There is therefore the risk that ionic species will also form during UV exposure, which then for their part are able to intervene oxidatively in the biochemical processes.

It is, moreover, known that undesired oxidation processes can arise in human and animal skin. The essay “Skin Diseases Associated with Oxidative Injury” in “Oxidative Stress in Dermatology”, p. 323 ff. (Marcel Decker Inc., New York, Basle, Hong Kong, editor: Jürgen Fuchs, Frankfurt, and Lester Packer, Berkeley/California) discusses such oxidative skin damage and its more probable causes.

Although antioxidants are primarily used as protection substances against the deterioration of the preparations in which they are present, antioxidants and/or free-radical scavengers can also be used in cosmetic or dermatological formulations in order to remedy or prevent oxidation reactions.

Thus, U.S. Pat. Nos. 4,144,325 and 4,248,861, and numerous other documents have already proposed the use of vitamin E—a substance with known antioxidative effect in light protection formulations—although here too the effect achieved nevertheless falls a long way short of the desired effect.

### SUMMARY OF THE INVENTION

An object of the present invention was therefore to avoid the disadvantages of the prior art and, in particular, to overcome the damage caused by environmental noxae permanently, in a sustained manner and without the risk of side effects, or to prevent them.

A further object of the invention was to create cosmetic, dermatological and pharmaceutical active ingredients and preparations and light protection formulations which serve for the prophylaxis and treatment of light-sensitive skin, in particular of photodermatoses, preferably of polymorphous light dermatosis.

To overcome these shortcomings was the object of the present invention.

It has surprisingly been found that the use of hops or hop-malt extracts in cosmetic or dermatological preparations for the treatment and prophylaxis of the symptoms of negative changes in the skin caused by the environment and/or of skin damage induced by UV and/or ozone and/or smog and/or smoking, and of inflammatory and degenerative skin conditions overcomes the disadvantages of the prior art.

Preferably, cosmetic or dermatological preparations according to the invention comprise 0.0005 to 50% by weight, preferably 0.01 to 20% by weight, particularly preferably 0.01 to 5% by weight, of hops or hop-malt extracts, in each case based on the total composition of the preparations.

### DETAILED DESCRIPTION OF PREFERRED EMBODIMENTS

Hop extract for the purposes of the present invention is obtained from botanically known types of hops (Humulus lupulus, Canabaceae family) by extraction.

An advantageous hop extract (hop cones and hop glands extracts) according to the invention typically comprises some or all of the following ingredients:
Hop bitter substances: humulones, lupulones

The bitter substances are monoacylphloroglucides with dimethylallyl side chains. Depending on the number of dimethylallyl side chains, a distinction is made between humulones (α-acids) with two dimethylallyl side chains, and lupulones (β-acids) with three dimethylallyl side chains.
Complete resins (methanol- and ether-soluble fraction)

A distinction is made between soft resins (hexane-soluble) and hard resins (hexane-insoluble).
Lipophilic hop extracts comprise 2-methyl-3-buten-2-ol, while hop extracts prepared with polar solvents are free from 2-methyl-3-buten-2-ol.
Polyphenols constitute 4 to 14% by weight of the dry substance depending on the type of hops, provenance and storage conditions.
Phenolcarboxylic acids, such as ferulic acid, gallic acid, caffeic acid, para-coumaric acid, para-hydroxybenzoic acid, protocatechuic acid, vanillic acid free and glycocidically bonded, chlorogenic acid, neochlorogenic acid.
Flavanones/chalcones (xanthohumol, isoxanthohumol, desmethylxanthohumol, 3-isoprenyl-2,4-dihydroxy-4,6-dimethoxychalcone and 2,6-dimethoxy-4,4-dihydroxychalcone)
Flavonols: astragalin, kaempferol and quercetin-3-glycosides
Catechins: catechin, epicatechin
Proanthocyanidins (leucocyanidin, leucodelphinidin)
Condensed tannins
Essential oils: terpene hydrocarbons, myrcene, humulene, β-caryophyllene, 2-undecanone
Phytosterols
Phytoestrogens
Glucides
Tannins

The composition of the hop extracts according to the invention, i.e., for example the complete resin amount, the soft resin/hard resin content, the content of α-acids (3 to 12% by weight) or β-acids (3 to 5% by weight), and the ratio of the humulone and lupulone homologs is dependent on the type of hop, the cultivation area, the harvest time, the drying and the storage of the hops.

The hop extracts for the purposes of the present invention may be water-soluble or oil-soluble.

Depending on the solvent and extraction process used, it is possible to produce water-soluble or oil-soluble hop extracts from hop cones and hop glands. For this, the fresh fruit (female flowers) or the dried plant is extracted for example by one of the following known processes:

1. An advantageous extractant for producing water-soluble extracts is, for example, but not exclusively, 1,2-propylene glycol:

1 part of dried, ground drug mixture is admixed with 10 parts of extractant and stirred at a gentle temperature over a fixed period; the mixture is then centrifuged. 1 part of dry drug material is again added to the product of centrifugation and the extraction operation is repeated. The following constituents are centrifuged off again and the plant extract is filtered under sterile conditions at a pressure of 5 atmospheres.

2. Oil-soluble extracts are preferably prepared, for example, but not exclusively, using sunflower oil, soybean oil or diisobutyl adipate.

3. In addition, depending on the desired ingredient, the following extraction processes known from the literature can be used advantageously, but not exclusively:
extraction with dichloromethane, with carbon dioxide
alcoholic or hydroalcoholic extraction
fluid extract: 1:1 in ethanol
tincture: 1:5 in ethanol 60%

The list of specified extraction processes is not of course intended to be limiting. Hop extracts according to the invention are obtainable by numerous methods known per se. New methods are in principle also conceivable for producing the extracts. It is essential in this connection that the hop extracts have the properties according to the invention.

The use of the active ingredient used according to the invention or of cosmetic or topical dermatological preparations with an effective content of active ingredient used according to the invention surprisingly enables effective treatment, but also prophylaxis
of deficient, sensitive or hypoactive skin conditions or deficient, sensitive or hypoactive conditions of skin appendages,
of certain degenerative symptoms of the skin (e.g. skin sagging, age spots, teleangiectases, disturbance of the osmolyte balance, of the natural skin pH and decrease in the epidermal and dermal cell layers, the constituents of connected tissue, the retinal cones and capillary vessels of the skin) and/or of the skin appendages,
of environmentally induced (caused by smoking, smog, reactive oxygen species, free radicals) and in particular, light-induced negative changes in the skin and the skin appendages,
of light-induced skin damage and UV-induced immunosuppression,
with reduced skin thickness,
of skin slackening and skin fatigue,
with disorders of the normal skin pH and the osmolyte balance,
of pigment disorders,
of itching,
of horny layer barrier disorders,
for changes in the transepidermal water loss and the normal moisture content of the skin,
for changes in the normal lipid peroxidations,
of hair loss and for improved hair growth,
for deviations from the normal cell-cell communication in the skin,
for changes in the normal fibroblast and keratinocyte proliferation,
for changes in the normal fibroblast and keratinocyte differentiation,
of inflammatory skin conditions, and atopic eczema, seborrhoeic eczema, polymorphous light dermatosis, psoriasis, vitiligo.

The active ingredient used according to the invention or cosmetic or topical dermatological preparations with an active content of active ingredient used according to the invention, however, surprisingly serve
to calm sensitive or irritated skin,
to maintain normal collagen, hyaluronic acid, elastin and glycosaminoglycan homeostasis,
for increased activation of proteolytic enzymes in the skin, such as, for example, metalloproteinases,
to stimulate intracellular DNA synthesis, in particular in deficient or hypoactive skin conditions,
for wound healing disorders,
for increasing cell renewal and regeneration of the skin,
for increasing the skin's own protective and repair mechanisms (for example for dysfunctional enzymes, DNA, lipids, proteins),
for the pre- and post-treatment in cases of topical application of laser and abrasive treatments, which serve, for example, to reduce skin wrinkles and scars, to counteract the resulting skin irritations and to promote the regeneration processes in the damaged skin.

According to the invention, it is extremely advantageous to use the active ingredient according to the invention or cosmetic or topical dermatological preparations with an effective content of active ingredient used according to the invention for the cosmetic or dermatological treatment or prophylaxis of undesired skin conditions.

The active ingredient used according to the invention can advantageously be incorporated into customary cosmetic and dermatological preparations, which may be present in various forms. As well as one or more oil phases (to which the cardiolipin is preferably incorporated), the preparations for the purposes of the present invention may preferably additionally comprise one or more water phases and be present, for example, in the form of W/O, O/W, multiple (W/O/W, O/W/O) emulsions. Such formulations can preferably also be a microemulsion, a solid emulsion (i.e. an emulsion which is stabilized by solids, e.g. a Pickering emulsion phase), a sprayable emulsion, a hydrodispersion or lipodispersion.

In addition, the active ingredient used according to the invention can advantageously be incorporated into a solution, a gel, a solid stick or else an aerosol.

Preferably, the emulsions according to the invention are O/W emulsions.

Particularly advantageous preparations are also obtained if antioxidants are used as additives or active ingredients. According to the invention, the preparations advantageously comprise one or more antioxidants. Favorable, but nevertheless optional antioxidants which may be used are all antioxidants customary or suitable for cosmetic and/or dermatological applications.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, β-carotene, lycopene) and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, y-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of benzoin resin, rutinic acid and derivatives thereof, ferulic acid and derivatives thereof, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of these listed active ingredients which are suitable according to the invention.

For the purposes of the present invention, water-soluble antioxidants, such as, for example, vitamins, e.g. ascorbic acid and derivatives thereof, can be used particularly advantageously.

The amount of antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or derivatives thereof are the antioxidant(s), it is advantageous to choose their respective concentrations from the range from 0.001 to 10% by weight, based on the total weight of the formulation.

If vitamin A or vitamin A derivatives, or carotenes or derivatives thereof are the antioxidant(s), it is advantageous to choose their respective concentrations from the range from 0.001 to 10% by weight, based on the total weight of the formulation.

It is in this connection likewise advantageous to add the active ingredient used according to the invention as additive to preparations which already comprise other active ingredients for other purposes.

According to the invention, further active ingredients (one or more compounds) can also very advantageously be chosen from the group of lipophilic active ingredients, in particular from the following group: alpha-lipoic acid, phytoene, D-biotin, coenzyme Q10, alpha-glucosylrutin, carnitine, carnosine, isoflavone, creatine, taurine.

It is also advantageous, although of course not obligatory, to present the active ingredient according to the invention in encapsulated form, e.g. in collagen matrices and other customary encapsulation materials, e.g. as cellulose encapsulations, in gelatin, wax matrices or liposomally encapsulated. In particular, wax matrices as are described in DE-A 43 08 282 have proven favorable. Particularly advantageous encapsulation forms for the purposes of the present invention are also cyclodextrin complexes of cardiolipin.

It may also be advantageous to encapsulate the active ingredient according to the invention e.g. as so-called solid lipid nanoparts using molten waxes, which may be chosen, inter alia, but not exclusively, from the group of ester waxes, triglyceride waxes or hydrocarbon waxes. In addition, it may be advantageous to encapsulate the active ingredients according to the invention in polymers, e.g. in particles based on highly crosslinked polymethacrylates and/or cellulose triacetates and/or as core/shell particles with a shell of poly(oxymethylurea), nylon, polyamides, polyurethane, polyesters, gelatin and polyolefins.

The prophylaxis and the cosmetic or dermatological treatment with the active ingredient used according to the invention or with the cosmetic or topical dermatological preparations with an effective content of active ingredient used according to the invention takes place in the customary manner, namely by applying the active ingredient used according to the invention or the cosmetic or topical dermatological preparations with an effective content of active ingredient used according to the invention to the affected areas of skin.

Emulsions according to the invention for the purposes of the present invention, e.g. in the form of a cream, a lotion, a cosmetic milk, are advantageous and comprise e.g. fats, oils, waxes and/or other fatty substances, and water and one or more emulsifiers, as are customarily used for such a type of formulation.

It is also possible and advantageous for the purposes of the present invention to add the active ingredient used according to the invention to aqueous systems or surfactant preparations for cleansing the skin and the hair.

It is of course known to the person skilled in the art that complex cosmetic compositions are in most cases inconceivable without the customary auxiliaries and additives. These include, for example, consistency-imparting agents, fillers, perfume, dyes, emulsifiers, additional active ingredients, such as vitamins or proteins, light protection agents, stabilizers, insect repellents, alcohol, water, salts, antimicrobially, proteolytically or keratolytically effective substances etc.

Corresponding requirements apply mutatis mutandis to the formulation of medicinal preparations.

Medicinal topical compositions for the purposes of the present invention generally comprise one or more medicaments in an effective concentration. For the sake of simplicity, for a clear distinction between cosmetic and medicinal use and corresponding products, reference is made to the legal provisions of the Federal Republic of Germany (e.g. Cosmetics Directive, Food and Drugs Act).

Accordingly, cosmetic or topical dermatological compositions for the purposes of the present invention can, depending on their formulation, be used, for example, in the form of skin protection cream, cleansing milk, sunscreen lotion, nutrient cream, day or night cream etc. It is in some cases possible and advantageous to use the compositions according to the invention as a basis for pharmaceutical formulations.

For the purposes of the present invention, it is also advantageous to provide cosmetic and dermatological preparations whose main purpose is not protection against sunlight, but which nevertheless have a content of UV protection substances. Thus, for example, UV-A and/or UV-B filter substances are usually incorporated into day creams or make-up products. UV protection substances, like antioxidants, and, if desired, preservatives, also constitute effective protection of the preparations themselves against spoilage. Also favorable are cosmetic and dermatological preparations in the form of a sunscreen.

Accordingly, for the purposes of the present invention, as well as comprising one or more active substance(s) according to the invention, the preparations additionally comprise at least one further UV-A and/or UV-B filter substance. The formulations may, although not necessarily, optionally also comprise one or more organic and/or inorganic pigments as UV filter substances which may be present in the water and/or oil phase.

Preferred inorganic pigments are metal oxides and/or other metal compounds which are insoluble or virtually insoluble in water, in particular oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals and mixtures of such oxides.

For the purposes of the present invention, such pigments may advantageously be surface-treated (“coated”), the intention being to form or retain, for example, an amphiphilic or hydrophobic character. This surface treatment can consist in providing the pigments with a thin hydrophobic layer by processes known per se.

Advantageous according to the invention are e.g. titanium dioxide pigments which have been coated with octylsilanol. Suitable titanium dioxide particles are available under the trade name T805 from Degussa. Also particularly advantageous are TiO₂ pigments coated with aluminum stearate, e.g. those available under the trade name MT 100 T from TAYCA.

A further advantageous coating of the inorganic pigments consists of dimethylpolysiloxane (also: dimethicone), a mixture of completely methylated, linear siloxane polymers which have been terminally blocked with trimethylsiloxy units. Particularly advantageous for the purposes of the present invention are zinc oxide pigments which have been coated in this way.

Also advantageous is a coating of the inorganic pigments with a mixture of dimethylpolysiloxane, in particular dimethylpolysiloxane having an average chain length of from 200 to 350 dimethylsiloxane units, and silica gel, which is also referred to as simethicone. In particular, it is advantageous for the inorganic pigments to be additionally coated with aluminum hydroxide or aluminum oxide hydrate (also: alumina, CAS No.: 1333-84-2). Particularly advantageous are titanium dioxides which have been coated with simethicone and alumina, it also being possible for the coating to comprise water. An example thereof is the titanium dioxide available under the trade name Eusolex T2000 from Merck.

An advantageous organic pigment for the purposes of the present invention is 2,2′-methylenebis(6-(2H-benzotriazol-2-yl)-4-(1,1,3,3-tetramethylbutyl)phenol) [INCI: bisoctyltriazole], which is available under the trade name Tinosorb® M from CIBA-Chemikalien GmbH.

Preparations according to the invention advantageously comprise substances which absorb UV radiation in the UV-A and/or UV-B range, the total amount of filter substances being, for example, from 0.1% by weight to 30% by weight, preferably from 0.5 to 20% by weight, in particular from 1.0 to 15.0% by weight, based on the total weight of the preparations, in order to provide cosmetic preparations which protect the hair and the skin from the entire range of ultraviolet radiation. They can also be used as sunscreens for the hair or the skin.

Advantageous UV-A filter substances for the purposes of the present invention are dibenzoylmethane derivatives, in particular 4-(tert-butyl)-4′-methoxydibenzoylmethane (CAS No. 70356-09-1), which is sold by Givaudan under the name Parsol® 1789 and by Merck under the trade name Eusolex® 9020.

Further advantageous UV-A filter substances are phenylene-1,4-bis(2-benzimidazyl)-3,3′-5,5′-tetrasulfonic acid and its salts, particularly the corresponding sodium, potassium or triethanolammonium salts, in particular phenylene-1,4-bis(2-benzimidazyl)-3,3′-5,5′-tetrasulfonic bis-sodium salt with the INCI name Bisimidazylate, which is available, for example, under the trade name Neo Heliopan AP from Haarmann & Reimer.

Also advantageous are 1,4-di(2-oxo-10-sulfo-3-bornylidenemethyl)benzene and salts thereof (in particular the corresponding 10-sulfato compounds, in particular the corresponding sodium, potassium or triethanolammonium salt), which is also referred to as benzene-1,4-di(2-oxo-3-bornylidenemethyl-10-sulfonic acid).

Advantageous UV filter substances for the purposes of the present invention are also broadband filters, i.e. filter substances which absorb both UV-A and also UV-B radiation.

Advantageous broadband filters or UV-B filter substances are, for example, bisresorcinyltriazine derivatives. Particular preference is given to 2,4-bis{[4-(2-ethylhexyloxy)-2-hydroxy]phenyl}-6-(4-methoxyphenyl)-1,3,5-triazine (INCI: Aniso Triazine), which is available under the trade name Tinosorb® S from CIBA-Chemikalien GmbH.

For the purposes of the present invention, particularly advantageous preparations which are characterized by high or very high UV-A protection preferably comprise two or more UV-A and/or broadband filters, in particular dibenzoylmethane derivatives [for example 4-(tert-butyl)-4′-methoxydibenzoylmethane], benzotriazole derivatives [for example 2,2′-methylenebis(6-(2H-benzotriazol-2-yl)-4-(1,1,3,3-tetramethylbutyl)phenol)], phenylene-1,4-bis(2-benzimidazyl)-3,3′-5,5′-tetrasulfonic acid and/or its salts, 1,4-di(2-oxo-10-sulfo-3-bornylidenemethyl)benzene and/or salts thereof and/or 2,4-bis{[4-(2-ethylhexyloxy)-2-hydroxy]phenyl}-6-(4-methoxyphenyl)-1,3,5-triazine, in each case individually or in any combinations with one another.

Other UV filter substances, which have the structural formula 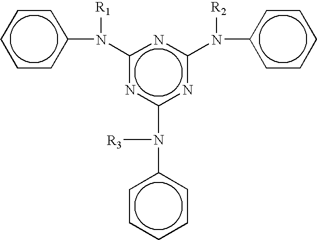
are also advantageous UV filter substances for the purposes of the present invention, for example the s-triazine derivatives described in European laid-open specification EP 570 838 A1, whose chemical structure is expressed by the generic formula 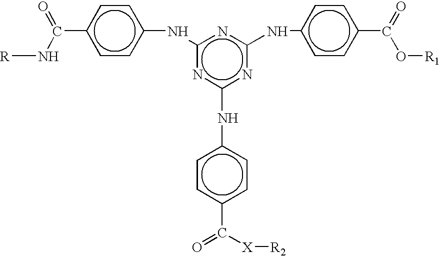
where
R is a branched or unbranched C₁-C₁₈-alkyl radical, a C₅-C₁₂-cycloalkyl radical, optionally substituted with one or more C₁-C₄-alkyl groups,
X is an oxygen atom or an NH group,
R₁ is a branched or unbranched C₁-C₁₋₈-alkyl radical, a C₅-C₁₂-cycloalkyl radical, optionally substituted by one or more C₁-C₄-alkyl groups, or a hydrogen atom, an alkali metal atom, an ammonium group or a group of the formula 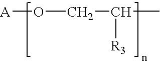
   in which
A is a branched or unbranched C₁-C₁₈-alkyl radical, a C₅-C₁₂-cycloalkyl or aryl radical, optionally substituted by one or more C₁-C₄-alkyl groups,
R₃ is a hydrogen atom or a methyl group,
n is a number from 1 to 10,
R₂ is a branched or unbranched C₁-C₁₈-alkyl radical, a C₅-C₁₂-cycloalkyl radical, optionally substituted by one or more C₁-C₄-alkyl groups, when X is the NH group, and
   a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula 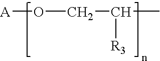
   in which
A is a branched or unbranched C₁-C₁₈-alkyl radical, a C₅-C₁₂-cycloalkyl or aryl radical, optionally substituted by one or more C₁-C₄-alkyl groups,
R₃ is a hydrogen atom or a methyl group,
n is a number from 1 to 10,
   when X is an oxygen atom.

A particularly preferred UV filter substance for the purposes of the present invention is also an unsymmetrically substituted s-triazine, the chemical structure of which is expressed by the formula 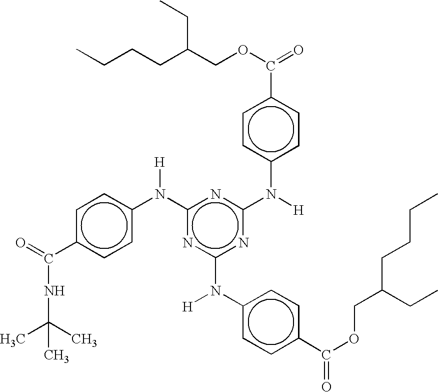
and which is also referred to below as dioctylbutylamidotriazone (INCI: Dioctylbutamidotriazone), and is available under the trade name UVASORB HEB from Sigma 3V.

Also advantageous for the purposes of the present invention is a symmetrically substituted s-triazine, tris(2-ethylhexyl) 4,4′,4″-(1,3,5-triazine-2,4,6-triyltriimino)trisbenzoate, synonym: 2,4,6-tris[anilino-(p-carbo-2′-ethyl-1′-hexyloxy)]-1,3,5-triazine (INCI: Octyl Triazone), which is marketed by BASF Aktiengesellschaft under the trade name UVINUL® T 150.

European laid-open specification 775 698 also describes preferred bisresorcinyltriazine derivatives, the chemical structure of which is expressed by the generic formula 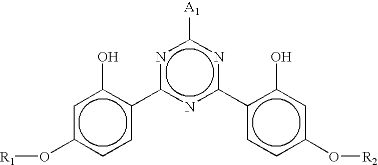
where R₁, R₂ and A₁ represent very different organic radicals.

Also advantageous for the purposes of the present invention are 2,4-bis{[4-(3-sulfonato)-2-hydroxypropyloxy)-2-hydroxy]phenyl}-6-(4-methoxyphenyl)-1,3,5-triazine sodium salt, 2,4-bis{[4-(3-(2-propyloxy)-2-hydroxypropyloxy)-2-hydroxy]phenyl}-6-(4-methoxyphenyl)-1,3,5-triazine, 2,4-bis{[4-(2-ethylhexyloxy)-2-hydroxy]phenyl}-6-[4-(2-methoxyethylcarboxyl)phenylamino]-1,3,5-triazine, 2,4-bis{[4-(3-(2-propyloxy)-2-hydroxypropyloxy)-2-hydroxy]phenyl}-6-[4-(2-ethylcarboxyl)phenylamino]-1,3,5-triazine, 2,4-bis{[4-(2-ethylhexyloxy)-2-hydroxy]phenyl}-6-(1-methylpyrrol-2-yl)-1,3,5-triazine, 2,4-bis{[4-tris(trimethylsiloxysilylpropyloxy)-2-hydroxy]phenyl}-6-(4-methoxyphenyl)-1,3,5-triazine, 2,4-bis{[4-(2″-methylpropenyloxy)-2-hydroxy]phenyl}-6-(4-methoxyphenyl)-1,3,5-triazine and 2,4-bis{[4-(1′,1′,1′,3′,5′,5′,5′-heptamethylsiloxy-2″-methylpropyloxy)-2-hydroxy]phenyl}-6-(4-methoxyphenyl)-1,3,5-triazine.

An advantageous broadband filter for the purposes of the present invention is 2,2′-methylenebis(6-(2H-benzotriazol-2-yl)-4-(1,1,3,3-tetramethylbutyl)phenol) and is available under the trade name Tinosorb® M from CIBA-Chemikalien GmbH.

Another advantageous broadband filter for the purposes of the present invention is 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) having the INCI name Drometrizole Trisiloxane.

The UV-B and/or broadband filters can be oil-soluble or water-soluble. Examples of advantageous oil-soluble UV-B and/or broadband filter substances are:
3-benzylidenecamphor derivatives, preferably 3-(4-methylbenzylidene)camphor, 3-benzylidenecamphor;
4-aminobenzoic acid derivatives, preferably 2-ethylhexyl 4-(dimethylamino)benzoate, amyl 4-(dimethylamino)benzoate;
2,4,6-trianilino(p-carbo-2′-ethyl-1′-hexyloxy)-1,3,5-triazine;
esters of benzalmalonic acid, preferably di(2-ethylhexyl) 4-methoxybenzalmalonate,
esters of cinnamic acid, preferably 2-ethylhexyl 4-methoxycinnamate, isopentyl 4-methoxycinnamate;
derivates of benzophenone, preferably 2-hydroxy-4-methoxybenzophenone, 2-hydroxy-4-methoxy-4′-methylbenzophenone, 2,2′-dihydroxy-4-methoxybenzophenone
and UV filters bonded to polymers.

Examples of advantageous water-soluble UV-B and/or broadband filter substances are:
salts of 2-phenylbenzimidazole-5-sulfonic acid, such as its sodium, potassium or its triethanolammonium salt, and also the sulfonic acid itself;
sulfonic acid derivatives of 3-benzylidenecamphor, such as, for example, 4-(2-oxo-3-bornylidenemethyl) benzenesulfonic acid, 2-methyl-5-(2-oxo-3-bornylidenemethyl)sulfonic acid and salts thereof.

A further light protection filter substance which can be used advantageously according to the invention is ethylhexyl 2-cyano-3,3-diphenylacrylate (octocrylene), which is available from BASF under the name Uvinul® N 539.

It can also be of considerable advantage to use polymer-bonded or polymeric UV filter substances in the preparations according to the present invention, in particular those described in WO-A-92/20690.

In some instances, it can also be advantageous to incorporate further UV-A and/or UV-B filters in accordance with the invention into cosmetic or dermatological preparations, for example certain salicylic acid derivatives, such as 4-isopropylbenzyl salicylate, 2-ethylhexyl salicylate (=octyl salicylate), homomenthyl salicylate.

The list of given UV filters which can be used for the purposes of the present invention is, of course, not intended to be limiting.

The preparations according to the invention advantageously comprise the substances which absorb UV radiation in the UV-A and/or UV-B region in a total amount of, for example, 0.1% by weight to 30% by weight, preferably 0.5 to 20% by weight, in particular 1.0 to 15.0% by weight, in each case based on the total weight of the preparations, in order to provide cosmetic preparations which protect the hair or the skin from the entire range of ultraviolet radiation. They can also be used as sunscreens for the hair or the skin.

The cosmetic and dermatological preparations according to the invention can comprise cosmetic active ingredients, auxiliaries and/or additives, as are customarily used in such preparations, e.g. antioxidants, preservatives, bactericides, perfumes, antifoams, dyes, pigments which have a coloring action, thickeners, surface-active substances, emulsifiers, softening, moisturizing and/or humectant substances, fats, oils, waxes or other customary constituents of a cosmetic or dermatological formulation, such as alcohols, polyols, polymers, foam stabilizers, electrolytes, organic solvents or silicone derivatives.

If the cosmetic or dermatological preparation for the purposes of the present invention is a solution or emulsion or dispersion, the solvents which may be used are:
water or aqueous solutions;
oils, such as triglycerides of capric acid or of caprylic acid, but preferably castor oil;
fats, waxes and other natural and synthetic fatty bodies, preferably esters of fatty acids with alcohols of low carbon number, e.g. with isopropanol, propylene glycol or glycerol, or esters of fatty alcohols with alkanoic acids of low carbon number or with fatty acids;
alcohols, diols or polyols of low carbon number, and ethers thereof, preferably ethanol, isopropanol, propylene glycol, glycerol, ethylene glycol, ethylene glycol monoethyl and monobutyl ether, propylene glycol monomethyl, monoethyl and monobutyl ether, diethylene glycol monomethyl or monoethyl ether and analogous products.

In particular, mixtures of the abovementioned solvents are used. In the case of alcoholic solvents, water may be a further constituent.

The oil phase of the emulsions, oleogels or hydrodispersions or lipodispersions for the purposes of the present invention is advantageously chosen from the group of esters of saturated and/or unsaturated, branched and/or unbranched alkanecarboxylic acids with a chain length of from 3 to 30 carbon atoms and saturated and/or unsaturated, branched and/or unbranched alcohols with a chain length of from 3 to 30 carbon atoms, from the group of esters of aromatic carboxylic acids and saturated and/or unsaturated, branched and/or unbranched alcohols with a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyidodecyl palmitate, oleyl oleate, oleyl erucate, erucyl oleate, erucyl erucate, and synthetic, semisynthetic and natural mixtures of such esters, e.g. jojoba oil.

In addition, the oil phase can advantageously be chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids with a chain length of from 8 to 24, in particular 12-18, carbon atoms. The fatty acid triglycerides can, for example, advantageously be chosen from the group of synthetic, semisynthetic and natural oils, e.g. olive oil, sunflower oil, soybean oil, groundnut oil, rapeseed oil, almond oil, palm oil, coconut oil, palm kernel oil and the like.

Any mixtures of such oil and wax components can also be used advantageously for the purposes of the present invention. It may in some instances also be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride and dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate are particularly advantageous.

Of the hydrocarbons, paraffin oil, squalane and squalene are to be used advantageously for the purposes of the present invention.

The oil phase can advantageously also have a content of cyclic or linear silicone oils or consist entirely of such oils, although it is preferred to use an additional content of other oil phase components apart from the silicone oil or the silicone oils.

Advantageously, cyclomethicone (octamethylcyclotetrasiloxane) is used as silicone oil to be used according to the invention. However, other silicone oils are also used advantageously for the purposes of the present invention, for example hexamethylcyclotrisiloxane, polydimethylsiloxane, poly(methylphenylsiloxane).

Mixtures of cyclomethicone and isotridecyl isononanoate, and of cyclomethicone and 2-ethylhexyl isostearate are also particularly advantageous.

The aqueous phase of the preparations according to the invention advantageously optionally comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, glycerol, and in particular one or more thickeners, which may be chosen advantageously from the group consisting of silicon dioxide, aluminum silicates, polysaccharides or derivatives thereof, e.g. hyaluronic acid, xanthan gum, hydroxypropylmethylcellulose, particularly advantageously from the group of polyacrylates, preferably a polyacrylate from the group of so-called Carbopols, for example Carbopol grades 980, 981, 1382, 2984, 5984, in each case individually or in combination.

Gels used according to the invention usually comprise alcohols of low carbon number, e.g. ethanol, isopropanol, 1,2-propanediol, glycerol and water or an abovementioned oil in the presence of a thickener which, in the case of oily-alcoholic gels, is preferably silicon dioxide or an aluminum silicate, and in the case of aqueous-alcoholic or alcoholic gels is preferably a polyacrylate.

Solid sticks comprise e.g. natural or synthetic waxes, fatty alcohols or fatty acid esters.

Customary base substances which are suitable for use as cosmetic sticks for the purposes of the present invention are liquid oils (e.g. paraffin oils, castor oil, isopropyl myristate), semisolid constituents (e.g. petroleum jelly, lanolin), solid constituents (e.g. beeswax, ceresin and microcrystalline waxes or ozokerite), and high-melting waxes (e.g. carnauba wax, candellila wax).

Propellants which can be used for cosmetic and/or dermatological preparations which can be sprayed from aerosol containers for the purposes of the present invention are the customary known readily volatile, liquefied propellants, for example hydrocarbons (propane, butane, isobutane), which can be used on their own or in a mixture with one another. Compressed air can also be used advantageously.

The person skilled in the art of course knows that there are nontoxic propellant gases which would in principle be suitable for realizing the present invention in the form of aerosol preparations, but which nevertheless should be omitted due to their unacceptable impact on the environment or other accompanying circumstances, in particular fluorocarbons and chlorofluorocarbons (CFCs).

Cosmetic preparations for the purposes of the present invention may also be in the form of gels which, as well as an effective content of the active ingredient according to the invention and solvents customarily used therefor, preferably water, also comprise organic thickeners, e.g. gum arabic, xanthan gum, sodium alginate, cellulose derivatives, preferably methylcellulose, hydroxymethylcellulose, hydroxyethylcellulose, hydroxypropylcellulose, hydroxypropylmethylcellulose or inorganic thickeners, e.g. aluminum silicates, such as, for example bentonite, or a mixture of polyethylene glycol and polyethylene glycol stearate or distearate. The thickener is present in the gel e.g. in an amount between 0.1 and 30% by weight, preferably between 0.5 and 15% by weight.

The examples below are intended to illustrate the present invention.

### 1. PIT Emulsions

| | 1 | 2 | 3 | 4 | 5 |
|---|---|---|---|---|---|
| Glycerol monostearate, self-emulsifying | 0.50 | | 3.00 | 2.00 | 4.00 |
| Polyoxyethylene(12) cetylstearyl ether | | 5.00 | | 1.00 | 1.50 |
| Polyoxyethylene(20) cetylstearyl ether | | | | 2.00 | |
| Polyoxyethylene(30) cetylstearyl ether | 5.00 | | 1.00 | | |
| Stearyl alcohol | | | 3.00 | | 0.50 |
| Cetyl alcohol | 2.50 | 1.00 | | 1.50 | |
| 2-Ethylhexyl methoxycinnamate | | | | 5.00 | 8.00 |
| 2,4-Bis(4-(2-ethylhexyloxy)-2- | | 1.50 | | 2.00 | 2.50 |
| hydroxy)phenyl)-6-(4-methoxyphenyl)- | | | | | |
| (1,3,5)-triazine | | | | | |
| 1-(4-tert-Butylphenyl)-3-(4- | | | 2.00 | | |
| methoxyphenyl)-1,3-propanedione | | | | | |
| Diethylhexylbutamidotriazone | 1.00 | 2.00 | | 2.00 | |
| Ethylhexyltriazone | 4.00 | | 3.00 | 4.00 | |
| 4-Methylbenzylidenecamphor | | 4.00 | | | 2.00 |
| Octocrylene | | 4.00 | | | 2.50 |
| Phenylene-1,4-bis(monosodium, 2- | | | 0.50 | | 1.50 |
| benzimidazyl-5,7-disulfonic acid | | | | | |
| Phenylbenzimidazolsulfonic acid | 0.50 | | | 3.00 | |
| C12-15 alkyl benzoate | | 2.50 | | | 5.00 |
| Titanium dioxide | 0.50 | 1.00 | | 3.00 | 2.00 |
| Zinc oxide | 2.00 | | 3.00 | 0.50 | 1.00 |
| Dicaprylyl ether | | | 3.50 | | |
| Butylene glycol dicaprylate/dicaprate | 5.00 | | | 6.00 | |
| Dicaprylyl carbonate | | | 6.00 | | 2.00 |
| Dimethicone polydimethylsiloxane | | 0.50 | 1.00 | | |
| Phenylmethylpolysiloxane | 2.00 | | | 0.50 | 0.50 |
| Shea butter | | 2.00 | | | 0.50 |
| PVP hexadecene copolymer | 0.50 | | | 0.50 | 1.00 |
| Glycerol | 3.00 | 7.50 | 5.00 | 7.50 | 2.50 |
| Tocopherol acetate | 0.50 | | 0.25 | | 1.00 |
| Hops or hop-malt extract | 0.10 | 0.50 | 1.00 | 0.20 | 0.10 |
| Alpha-glucosylrutin | 0.10 | | 0.20 | | |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad. 100 | ad. 100 | ad. 100 | ad. 100 | ad. 100 |

### 2. Examples of O/W Cream

| Examples | 1 | 2 | 3 | 4 | 5 |
|---|---|---|---|---|---|
| Glyceryl stearate citrate | | | 2.00 | | 2.00 |
| Glyceryl stearate, self-emulsifying | 4.00 | 3.00 | | | |
| PEG-40 stearate | 1.00 | | | | |
| Polyglyceryl-3 methylglucose distearate | | | | 3.00 | |
| Sorbitan stearate | | | | | 2.00 |
| Stearic acid | | 1.00 | | | |
| Polyoxyethylene(20) cetylstearyl ether | | | | | |
| Stearyl alcohol | | | 5.00 | | |
| Cetyl alcohol | 3.00 | 2.00 | | 3.00 | |
| Cetylstearyl alcohol | | | | | 2.00 |
| C12-15 alkyl benzoate | | | | | |
| Caprylic/capric triglyceride | 5.00 | 3.00 | 4.00 | 3.00 | 3.00 |
| Octyldodecanol | | | 2.00 | | 2.00 |
| Dicaprylyl ether | | 4.00 | | 2.00 | 1.00 |
| Paraffinum liquidum | 5.00 | 2.00 | | 3.00 | |
| Titanium dioxide | | | 1.00 | | |
| 4-Methylbenzylidenecamphor | | | 1.00 | | |
| 1-(4-tert-Butylphenyl)-3-(4- | | | 0.50 | | |
| methoxyphenyl)-1,3-propanedione | | | | | |
| Hops or hop-malt extract | 0.20 | 0.50 | 0.10 | 1.00 | 0.30 |
| Tocopherol | 0.1 | | | | 0.20 |
| Biotin | | | 0.05 | | |
| Ethylenediaminetetraacetic acid trisodium | 0.1 | | 0.10 | 0.1 | |
| Preservative | q.s. | q.s. | q.s. | q.s. | q.s. |
| Xanthan gum | | | | | |
| Polyacrylic acid | 3.00 | 0.1 | | 0.1 | 0.1 |
| Sodium hydroxide solution 45% | q.s. | q.s. | q.s. | q.s. | q.s. |
| Glycerol | 5.00 | 3.00 | 4.00 | 3.00 | 3.00 |
| Butylene glycol | | 3.00 | | | |
| Perfume | q.s. | q.s. | q.s. | q.s. | q.s. |
| Water | ad. 100 | ad. 100 | ad. 100 | ad. 100 | ad. 100 |

### 3. Examples of O/W Cream

| Examples | 1 | 2 | 3 | 4 | 5 |
|---|---|---|---|---|---|
| Glyceryl stearate citrate | | 2.00 | 2.00 | | |
| Glyceryl stearate, self-emulsifying | 5.00 | | | | |
| Stearic acid | | | | 2.50 | 3.50 |
| Stearyl alcohol | 2.00 | | | | |
| Cetyl alcohol | | | | 3.00 | 4.50 |
| Cetylstearyl alcohol | | 3.00 | 1.00 | | 0.50 |
| C12-15 alkyl benzoate | | 2.00 | 3.00 | | |
| Caprylic/capric triglyceride | 2.00 | | | | |
| Octyldodecanol | 2.00 | 2.00 | | 4.00 | 6.00 |
| Dicaprylyl ether | | | | | |
| Paraffinum liquidum | | 4.00 | 2.00 | | |
| Cyclic dimethylpolysiloxane | | | | 0.50 | 2.00 |
| Dimethiconepolydimethylsiloxane | 2.00 | | | | |
| Titanium dioxide | 2.00 | | | | |
| 4-Methylbenzylidenecamphor | 1.00 | | | | 1.00 |
| 1-(4-tert-Butylphenyl)-3-(4- | 0.50 | | | | 0.50 |
| methoxyphenyl)-1,3-propanedione | | | | | |
| Hops or hop-malt extract | 0.20 | 0.70 | 0.25 | 1.00 | 0.40 |
| Tocopherol | | | | | 0.05 |
| Ethylenediaminetetraacetic acid trisodium | | | 0.20 | | 0.20 |
| Preservative | q.s. | q.s. | q.s. | q.s. | q.s. |
| Xanthan gum | | | 0.20 | | |
| Polyacrylic acid | 0.15 | 0.1 | | 0.05 | 0.05 |
| Sodium hydroxide solution 45% | q.s. | q.s. | q.s. | q.s. | q.s. |
| Glycerol | 3.00 | | 3.00 | 5.00 | 3.00 |
| Butylene glycol | | 3.00 | | | |
| Ethanol | | 3.00 | | 3.00 | |
| Perfume | q.s. | q.s. | q.s. | q.s. | q.s. |
| Water | ad. 100 | ad. 100 | ad. 100 | ad. 100 | ad. 100 |

### 4. Examples of W/O Emulsions

| | 1 | 2 | 3 | 4 | 5 |
|---|---|---|---|---|---|
| Cetyldimethicone copolyol | | 2.50 | | 4.00 | |
| Polyglyceryl-2 dipolyhydroxystearate | 5.00 | | | | 4.50 |
| PEG-30 dipolyhydroxystearate | | | 5.00 | | |
| 2-Ethylhexyl methoxycinnamate | | 8.00 | | 5.00 | 4.00 |
| 2,4-Bis(4-(2-ethylhexyloxy)-2- | 2.00 | 2.50 | | 2.00 | 2.50 |
| hydroxy)phenyl)-6-(4-methoxyphenyl)- | | | | | |
| (1,3,5)-triazine | | | | | |
| 1-(4-tert-Butylphenyl)-3-(4- | | | 2.00 | 1.00 | |
| methoxyphenyl)-1,3-propanedione | | | | | |
| Diethylhexylbutamidotriazine | 3.00 | 1.00 | | | 3.00 |
| Ethylhexyltriazone | | | 3.00 | 4.00 | |
| 4-Methylbenzylidenecamphor | | 2.00 | | 4.00 | 2.00 |
| Octocrylene | 7.00 | 2.50 | 4.00 | | 2.50 |
| Diethylhexylbutamidotriazone | 1.00 | | | 2.00 | |
| Phenylene-1,4-bis(monosodium, 2- | 1.00 | 2.00 | 0.50 | | |
| benzimidazyl-5,7-disulfonic acid) | | | | | |
| Phenylbenzimidazolesulfonic acid | 0.50 | | | 3.00 | 2.00 |
| Titanium dioxide | | 2.00 | 1.50 | | 3.00 |
| Zinc oxide | 3.00 | 1.00 | 2.00 | 0.50 | |
| Paraffinum liquidum | | | 10.0 | | 8.00 |
| C12-15 alkyl benzoate | | | | 9.00 | |
| Dicaprylyl ether | 10.00 | | | | 7.00 |
| Butylene glycol dicaprylate/dicaprate | | | 2.00 | 8.00 | 4.00 |
| Dicaprylyl carbonate | 5.00 | | 6.00 | | |
| Dimethicone polydimethylsiloxane | | 4.00 | 1.00 | 5.00 | |
| Phenylmethylpolysiloxane | 2.00 | 25.00 | | | 2.00 |
| Shea butter | | | 3.00 | | |
| PVP hexadecene copolymer | 0.50 | | | 0.50 | 1.00 |
| Octoxyglycerol | | 0.30 | 1.00 | | 0.50 |
| Glycerol | 3.00 | 7.50 | | 7.50 | 2.50 |
| Glycine soya | | 1.00 | 1.50 | | |
| Magnesium sulfate | 1.00 | 0.50 | | 0.50 | |
| Magnesium chloride | | | 1.00 | | 0.70 |
| Tocopherol acetate | 0.50 | | 0.25 | | 1.00 |
| Hops or hop-malt extract | 0.10 | 0.60 | 1.00 | 1.00 | 0.80 |
| Preservative | q.s. | q.s. | q.s. | q.s. | q.s. |
| Ethanol | 3.00 | | 1.50 | | 1.00 |
| Perfume | q.s. | q.s. | q.s. | q.s. | q.s. |
| Water | ad. 100 | ad. 100 | ad. 100 | ad. 100 | ad. 100 |

### 5. Examples of W/O Emulsions

| | 6 | 7 |
|---|---|---|
| Polyglyceryl-2 dipolyhydroxystearate | 4.00 | 5.00 |
| PEG-30 dipolyhydroxystearate | | |
| Lanolin alcohol | 0.50 | 1.50 |
| Isohexadecane | 1.00 | 2.00 |
| Myristyl myristate | 0.50 | 1.50 |
| Petroleum jelly | 1.00 | 2.00 |
| 1-(4-tert-Butylphenyl)-3-(4-methoxyphenyl)-1,3- | 0.50 | 1.50 |
| propanedione | | |
| 4-Methylbenzylidenecamphor | 1.00 | 3.00 |
| Butylene glycol dicaprylate/dicaprate | 4.00 | 5.00 |
| Shea butter | | 0.50 |
| Butylene glycol | | 6.00 |
| Octoxyglycerol | | 3.00 |
| Glycerol | 5.00 | |
| Tocopherol acetate | 0.50 | 1.00 |
| Hops or hop-malt extract | 0.20 | 0.25 |
| Trisodium EDTA | 0.20 | 0.20 |
| Preservative | q.s. | q.s. |
| Ethanol | | 3.00 |
| Perfume | q.s. | q.s. |
| Water | ad. 100 | ad. 100 |

### 6. Examples of Hydrodispersions

| | 1 | 2 | 3 | 4 | 5 |
|---|---|---|---|---|---|
| Polyoxyethylene(20) cetylstearyl ether | 1.00 | | | 0.5 | |
| Cetyl alcohol | | | 1.00 | | |
| Sodium polyacrylate | | 0.20 | | 0.30 | |
| Acrylates/C10-30 alkyl acrylate | 0.50 | | 0.40 | 0.10 | 0.10 |
| crosspolymer | | | | | |
| Xanthan gum | | 0.30 | 0.15 | | 0.50 |
| 2-Ethylhexyl methoxycinnamate | | | | 5.00 | 8.00 |
| 2,4-Bis(4-(2-ethylhexyloxy)-2- | | 1.50 | | 2.00 | 2.50 |
| hydroxy)phenyl)-6-(4-methoxyphenyl)- | | | | | |
| (1,3,5)-triazine | | | | | |
| 1-(4-tert-Butylphenyl)-3-(4- | 1.00 | | 2.00 | | |
| methoxyphenyl)-1,3-propanedione | | | | | |
| Diethylhexylbutamidotriazone | | 2.00 | | 2.00 | 1.00 |
| Ethylhexyltriazone | 4.00 | | 3.00 | 4.00 | |
| 4-Methylbenzylidenecamphor | 4.00 | 4.00 | | | 2.00 |
| Octocrylene | | 4.00 | 4.00 | | 2.50 |
| Phenylene-1,4-bis(monosodium, 2- | 1.00 | | 0.50 | | 2.00 |
| benzimidazyl-5,7-disulfonic acid | | | | | |
| Phenylbenzimidazolesulfonic acid | 0.50 | | | 3.00 | |
| Titanium dioxide | 0.50 | | 2.00 | 3.00 | 1.00 |
| Zinc oxide | 0.50 | 1.00 | 3.00 | | 2.00 |
| C12-15 alkyl benzoate | 2.00 | 2.50 | | | |
| Dicaprylyl ether | | 4.00 | | | |
| Butyleneglycol dicaprylate/dicaprate | 4.00 | | 2.00 | 6.00 | |
| Dicaprylyl carbonate | | 2.00 | 6.00 | | |
| Dimethicone polydimethylsiloxane | | 0.50 | 1.00 | | |
| Phenylmethylpolysiloxane | 2.00 | | | 0.50 | 2.00 |
| Shea butter | | 2.00 | | | |
| PVP hexadecene copolymer | 0.50 | | | 0.50 | 1.00 |
| Octoxyglycerol | | | 1.00 | | 0.50 |
| Glycerol | 3.00 | 7.50 | | 7.50 | 2.50 |
| Glycine soya | | | 1.50 | | |
| Tocopherol acetate | 0.50 | | 0.25 | | 1.00 |
| Hops or hop-malt extract | 0.15 | 0.60 | 1.00 | 1.00 | 0.80 |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad. 100 | ad. 100 | ad. 100 | ad. 100 | ad. 100 |

### 7. Example (Gel Cream)

| | |
|---|---|
| Acrylate/C10-30 alkyl acrylate cross- | 0.40 |
| polymer | |
| Polyacrylic acid | 0.20 |
| Xanthan gum | 0.10 |
| Cetearyl alcohol | 3.00 |
| C12-15 alkyl benzoate | 4.00 |
| Caprylic/capric triglyceride | 3.00 |
| Cyclic dimethylpolysiloxane | 5.00 |
| Dimethicone polydimethylsiloxane | 1.00 |
| Hops or hop-malt extract | 0.20 |
| Glycerol | 3.00 |
| Sodium hydroxide | q.s. |
| Preservative | q.s. |
| Perfume | q.s. |
| Water | ad 100.0 |
| pH adjusted to 6.0 | |

### 8. Example (W/O Cream)

| | |
|---|---|
| Polyglyceryl-3 diisostearate | 3.50 |
| Glycerol | 3.00 |
| Polyglyceryl-2 dipolyhydroxystearate | 3.50 |
| Hops or hop-malt extract | 0.50 |
| Preservative | q.s. |
| Perfume | q.s. |
| Water | ad. 100.0 |
| Magnesium sulfate | 0.6 |
| Isopropyl stearate | 2.0 |
| Caprylyl ether | 8.0 |
| Cetearyl isononanoate | 6.0 |

### 9. Example (W/O/W Cream)

| | |
|---|---|
| Glyceryl stearate | 3.00 |
| PEG-100 stearate | 0.75 |
| Behenyl alcohol | 2.00 |
| Caprylic/capric triglyceride | 8.0 |
| Octyldodecanol | 5.00 |
| C₁₂₋₁₅ alkyl benzoate | 3.00 |
| Hops or hop-malt extract | 1.00 |
| Magnesium sulfate (MgSO4) | 0.80 |
| Ethylenediaminetetraacetic acid | 0.10 |
| Preservative | q.s. |
| Perfume | q.s. |
| Water | ad. 100.0 |
| pH adjusted to 6.0 | |

### 10. Example of W/O Stick

| | |
|---|---|
| PEG-45/dodecylglycol copolymer | 2.00 |
| Polyglyceryl-3 diisostearate | 2.00 |
| Caprylic/capric triglyceride | 4.00 |
| Cetearyl isononanoate | 4.00 |
| Butylene glycol dicaprylate/dicaprate | 5.00 |
| Ethylhexyl methoxycinnamate | 5.00 |
| Ethylhexyltriazone | 3.00 |
| Bisethylhexyloxyphenol | 2.50 |
| methoxyphenyltriazine | |
| Hombitec H | 2.00 |
| C20-40 alkyl stearate | 9.00 |
| Silica dimethylsilylate | 1.00 |
| Dimethicone | 0.50 |
| Glycerol | 10.0 |
| Hops or hop-malt extract | 0.20 |
| PVP/hexadecene copolymer | 0.50 |
| Tocopherol acetate | 1.00 |
| Preservative | q.s. |
| Perfume | q.s. |
| Water | ad. 100 |

### 11. Example of W/O Stick

| | |
|---|---|
| PEG-45/dodecylglycol copolymer | 2.00 |
| Polyglyceryl-3 diisostearate | 2.00 |
| Cetearyl isononoate | 15.00 |
| C20-40-alkyl stearate | 8.00 |
| Glycerol | 10.00 |
| Hops or hop-malt extract | 0.50 |
| Preservative | q.s. |
| Perfume | q.s. |
| Water | ad. 100.00 |

1. A cosmetic or dermatological preparation for the treatment or prophylaxis of skin damage symptoms, inflammatory skin symptoms or degenerative skin symptoms, said preparation comprising hops or a hop-malt extract.

2. The preparation as claimed in claim 1, comprising no more than 1% by weight of hops or hop-malt extracts, based on the total weight of the preparation.

3. The preparation as claimed in claim 1, further comprising one or more of the group consisting of alpha-lipoic acid, phyotene, D-biotin, coenzyme Q10, alpha-glucosylrutin, carnitine, carnosine, isoflavones, clover extracts, soybean extracts, creatine, taurine and derivatives thereof.

4. The preparation as claimed in claim 1, comprising a hop-malt extract, wherein the hop-malt extract comprises a methanol-soluble and ether-soluble fraction.

5. The preparation as claimed in claim 1, comprising a hop-malt extract, wherein the hop-malt extract comprises a hexane-soluble resin.

6. The preparation as claimed in claim 1, comprising a hop-malt extract, wherein the hop-malt extract comprises a hexane-insoluble resin.

7. The preparation as claimed in claim 1, comprising a hop-malt extract, wherein the hop-malt extract is lipophilic.

8. The preparation as claimed in claim 7, wherein the lipophilic hop-malt extract includes 2-methyl-3-buten-2-ol.

9. The preparation as claimed in claim 7, comprising a hop-malt extract, wherein the hop-malt extract is extracted from one or more of sunflower oil, soybean oil and diisobutyl adipate.

10. The preparation as claimed in claim 1, comprising a hop-malt extract, wherein the hop-malt extract is water-soluble.

11. The preparation as claimed in claim 10, wherein the water-soluble hop-malt extract is free of 2-methyl-3-buten-2-ol.

12. The preparation as claimed in claim 10, comprising a hop-malt extract, wherein the hop-malt extract extracted from 1,2-propylene glycol.

13. The preparation as claimed in claim 1, comprising a hop-malt extract, wherein the hop-malt extract comprises polyphenols.

14. The preparation as claimed in claim 1, comprising a hop-malt extract, wherein the hop-malt extract comprises phenolcarboxylic acids.

15. The preparation as claimed in claim 1, comprising a hop-malt extract, wherein the hop-malt extract comprises at least one compound selected from the group consisting of flavanones and chalcones.

16. The preparation as claimed in claim 1, comprising a hop-malt extract, wherein the hop-malt extract comprises flavonols.

17. The preparation as claimed in claim 1, comprising a hop-malt extract, wherein the hop-malt extract comprises catechins.

18. The preparation as claimed in claim 1, comprising a hop-malt extract, wherein the hop-malt extract comprises proanthocyanidins.

19. The preparation as claimed in claim 1, comprising a hop-malt extract, wherein the hop-malt extract comprises condensed tannins.

20. The preparation as claimed in claim 1, comprising a hop-malt extract, wherein the hop-malt extract comprises essential oils.

21. The preparation as claimed in claim 1, comprising a hop-malt extract, wherein the hop-malt extract comprises phytosterols.

22. The preparation as claimed in claim 1, comprising a hop-malt extract, wherein the hop-malt extract comprises phytoestrogens.

23. The preparation as claimed in claim 1, comprising a hop-malt extract, wherein the hop-malt extract comprises glucides.

24. The preparation as claimed in claim 1, comprising a hop-malt extract, wherein the hop-malt extract comprises flavonols.

25. The preparation as claimed in claim 1, comprising a hop-malt extract, wherein the hop-malt extract comprises 3% to 12% by weight α-acids based on the total weight of the hop-malt extract.

26. The preparation as claimed in claim 1, comprising a hop-malt extract, wherein the hop-malt extract comprises 3% to 5% by weight β-acids based on the total weight of the hop-malt extract.

27. The preparation as claimed in claim 1, comprising a hop-malt extract, wherein the hop-malt extract is extracted from hop cones and hop glands.

28. The preparation as claimed in claim 1, comprising a hop-malt extract, wherein the hop-malt extract is extracted using dichloromethane and carbon dioxide.

29. The preparation as claimed in claim 1, comprising a hop-malt extract, wherein the hop-malt extract is extracted using an alcoholic or hydroalcoholic extraction.

30. The preparation as claimed in claim 1, comprising a hop-malt extract, wherein the hop-malt extract is extracted using a fluid extract in a 1:1 ratio in ethanol.

31. The preparation as claimed in claim 1, comprising a hop-malt extract, wherein the hop-malt extract is extracted using a tincture having a 1:5 ratio in 60% ethanol.

32. The preparation as claimed in claim 1, said preparation provided as an oil-in-water emulsion.

33. A method for treating or preventing the symptoms of skin damage, inflammatory skin conditions or degenerative skin conditions, comprising the step of applying a cosmetic or dermatological preparation comprising hops or a hop-malt extract to the skin.

34. The method as claimed in claim 33, wherein said applying step comprises applying the preparation to the skin to treat or prevent the symptoms of degenerative skin conditions.

35. The method as claimed in claim 33, wherein said applying step comprises applying the preparation to the skin to prevent the symptoms of inflammatory skin conditions or degenerative skin conditions.

36. The method as claimed in claim 35, wherein the preparation includes from 0.01% to 5% by weight of hops or hop-malt extracts, based on the total weight of the preparation.

37. The method as claimed in claim 33, wherein said applying step comprises applying the preparation to the skin to treat the symptoms of skin damage, inflammatory skin conditions or degenerative skin conditions.

38. The method as claimed in claim 37, wherein the preparation includes from 0.01% to 5% by weight of hops or hop-malt extracts, based on the total weight of the preparation.

39. The method as claimed in claim 37, wherein said applying step comprises applying the preparation to the skin to treat the symptoms of skin damage caused by UV.

40. The method as claimed in claim 39, wherein the preparation includes from 0.01% to 5% by weight of hops or hop-malt extracts, based on the total weight of the preparation.

41. The method as claimed in claim 33, wherein said applying step comprises applying the preparation to the skin to prevent or treat the symptoms of skin damage caused by ozone, smog or smoking.

42. The method as claimed in claim 41, wherein the preparation includes from 0.01% to 5% by weight of hops or hop-malt extracts, based on the total weight of the preparation.

43. The method as claimed in claim 33, comprising no more than 1% by weight of hops or hop-malt extracts, based on the total weight of the preparation.

44. The method as claimed in claim 33, further comprising the step of preparing the preparation using a hop-malt extract that is oil-soluble.

45. The method as claimed in claim 44, wherein said preparing step comprises preparing the preparation using an extract that is extracted using one or more of sunflower oil, soybean oil and diisobutyl adipate.

46. The method as claimed in claim 33, further comprising the step of preparing the preparation using a hop-malt extract that is water-soluble.

47. The method as claimed in claim 46, wherein said preparing step comprises preparing the preparation using an extract that is extracted using 1,2-propylene glycol.
